(19) United States
(12) Patent Application Publication
(10) Pub. No.: US 2018/0311340 A1
(43) Pub. Date: Nov. 1, 2018

(54) NEW ADJUVANT AND VACCINE COMPOSITION CONTAINING THE SAME

(71) Applicant: CADILA HEALTHCARE LIMITED, Ahmedabad, Gujarat (IN)

(72) Inventors: Epifanio FICHERA, Ahmedabad (IN); Gaurav GUPTA, Ahmedabad (IN); Reinhard GLUECK, Ahmedabad (IN)

(21) Appl. No.: 15/768,776

(22) PCT Filed: Oct. 17, 2016
(86) PCT No.: PCT/IB2016/056213

§ 371 Date: Apr. 16, 2018

(30) Foreign Application Priority Data
Oct. 19, 2015 (IN) 3960/MUM/2015

(51) Int. Cl. A61K 39/39; C12N 7/00; A61K 39/145; A61K 39/12; A61P 31/16; A61P 31/20; A61P 33/06; A61K 39/015; A61K 39/00; A61P 9/00

(52) U.S. Cl. CPC A61K 39/39; A61K 2039/55566; A61K 39/145; A61K 39/12; A61P 31/16; A61P 31/20; A61P 33/06; A61K 39/015; A61K 39/0012; A61P 9/00; A61K 2039/55511; A61K 2039/55588; A61K 2039/55505; A61K 2039/55572; C12N 2760/16034; C12N 2760/16071; C12N 2760/00034; C12N 2760/00071; A61K 2039/70; A61K 2039/545; A61K 2039/5254; C12N 2710/20034; C12N 2710/20071; C12N 7/00

(57) ABSTRACT

The present invention relates to certain polyphenol(s) having adjuvant property that can be used for the vaccine preparation. Also, the current invention provides adjuvant system comprising said polyphenol(s) and delivery system such as an immunostimulating reconstituted influenza virosomes (IRIVs). The present invention illustrates the said polyphenol(s) or an adjuvant system comprising of such polyphenol(s) and IRIVs can provide better level of immune response against antigen of interest than conventional vaccine systems. The preferred polyphenol according to the present invention can be beta-sitosterol. Beta-sitosterol can be optionally combined with the known adjuvant(s) to enhance immune response.

### FIELD OF THE INVENTION

The present invention relates to certain polyphenol(s) having adjuvant property that can be used for the vaccine preparation. Also, the current invention provides adjuvant system comprising said polyphenol(s) and a delivery system such as an immunostimulating reconstituted influenza virosomes (IRIVs). The present invention illustrates the said polyphenol(s) or an adjuvant system comprising of such polyphenol(s) and IRIVs can provide better level of immune response against antigen of interest than conventional vaccine systems. The preferred polyphenol according to the present invention can be beta-sitosterol. Beta-sitosterol can be optionally combined with the known adjuvant(s) to enhance immune response.

### BACKGROUND OF THE INVENTION

Majority of vaccine antigens currently under investigation are composed of highly purified recombinant molecules or subunits of pathogens and as such they lack several features of the pathogens, including the inherent immunostimulatory property, and thus often do not elicit strong immune responses. Despite assessments of a large number of adjuvants, aluminum-based mineral salts (alum) remains the most used approved adjuvant for human vaccines. Alum has a good track record of safety and has been considered the adjuvant of choice for vaccination against infections that can be prevented by antibody response, and as such has been widely and successfully used in many licensed vaccines. However, some limitations of alum are also well known. Alum fails to confer adequate increase of antibody response to small-size peptides as well as certain vaccines such as typhoid fever and influenza vaccines. Notably, alum is known to be a poor adjuvant for induction of cytotoxic T cell immunity and T helper 1 (Thl) responses, required to combat several life-threatening infections (Vaccine 28 (2010) 2363-2366). Thus there is an imperative need to develop novel adjuvants to support the development of vaccines against pathogens that have so far been refractory to the traditional vaccination strategies and to overcome the limitations of the available licensed adjuvants.

Although a lot of efforts have been focused on the development of new adjuvants, which include mineral salts, detoxified toxins, lipopeptide, emulsions, cytokines, polysaccharides, nucleic acid etc., very few are presently approved for human use. The main drawbacks in the development of new adjuvant are related to undesired side effects which may be either localized or systemic, difficulty of manufacture, poor stability and high production costs.

Based on their dominant mechanisms of action, adjuvants can be divided into two classes: immunopotentiators and delivery systems Immunopotentiators activate innate immunity directly (e.g. cytokines) or through pattern recognition receptors (PRRs) (such as bacterial components), whereas delivery systems (e.g. microparticles and nanoparticles) concentrate the antigen and display antigens in repetitive patterns, target vaccine antigens to Antigen presenting cells (APCs) and help co-localize antigens and immunopotentiators. Thus, both immune-potentiators and delivery systems can serve to augment antigen-specific immune response in vivo.

The first adjuvant activity was discovered empirically in 1926 with diphtheria toxoid absorbed to alum. Since then, despite several decades of research, only few adjuvants have been licensed for the use in humans in the major markets. Most of adjuvants till now discovered, although evaluated as more potent of alum, are considered unsuitable for human use due to their local or systemic toxicity. Thus, one of the major challenges in adjuvant research is to gain potency while minimizing toxicity. The difficulty of achieving this objective is reflected in the fact that alum, which despite being initially discovered over 80 years ago, remains the dominant human adjuvant in use even today.

Here, the present invention provides selected polyphenol(s) having adjuvant property that can be used in vaccine preparation. These polyphenol(s) are preferably phytosterols. The current invention provides selected polyphenol preferably beta-sitosterol as an adjuvant for the preparation of vaccine against target antigen(s).

“Accumulating evidence suggests that select vitamins and a subclass of polyphenols that is flavonoids, collectively referred to as nutritive adjuvants have immunomodulating properties. Most vitamins and flavonoids have been used as dietary supplements for immune enhancement. Combinations of selected vitamins and a flavonoid formulated in a vegetable oil synergistically enhanced immune responses when co-administered with an antigen and given through mucosal (intranasal and sublingual) and systemic (intramuscular) routes. But, all phytosterols including flavonoids that are known to have immunomodulatory properties do not work as adjuvants. Some flavonoids possess immune-enhancing properties; other in vitro and in vivo studies on the very same flavonoids suggest immunosuppressive effects. Dietary supplementation of each selected vitamin and flavonoid may induce an immune-enhancing or immunosuppressive effect in combination.” (Expert Opin. Biol. Ther. (2011) 11(11):1501-1513). This document shows uncertainty of flavonoid with regard to their immunosuppressive or immune-enhancing properties. Furthermore, it is suggested to use flavonoids in combination with vitamins as a nutritive supplement.

In particular, the present invention provides novel vaccine composition containing target antigen and phytosterols as an adjuvant alone. Such novel vaccine composition according to the present invention provides surprisingly higher immune response against target antigen as compared to composition comprising target antigen with alum or other conventional adjuvant(s). Further, the present invention provides novel adjuvant system comprising said polyphenol(s) and a suitable delivery system such as immunostimulating reconstituted influenza virosomes (IRIVs).

One of the studies of apple polyphenol extract (APE) has provided evidence that co-administration of an optimal dose of APE dramatically mitigates Cholera Toxin (CT) toxicity without altering mucosal adjuvant activity of native CT to induce Ag-specific humoral immunity in the mucosal and systemic compartments of the mouse model. Findings showed that the biological effects of APE on adjuvanticity and toxicity of CT were dose-dependent. (Vaccine 27 (2009) 4808-4817) Again, it suggests using polyphenol extracted from apple fruit in combination with the cholera toxin adjuvant to mitigate toxicity of the cholera toxin. APE has not been evaluated as an adjuvant here.

WO 2005/117958 provides virosome preparations from an enveloped virus, in particular from influenza virus, containing an antigen from said virus, and a saponin adjuvant. In particular the invention provides a virosome preparation from influenza virus containing an influenza antigen QS21, optionally with a sterol. Suitable sterols include β-sitosterol, stigmasterol, ergosterol, ergocalciferol and cholesterol, which is preferred. These sterols are well known in the art, for example cholesterol is disclosed in the Merck Index, 11th Edn, page 341, as a naturally occurring sterol found in animal fat. Said composition has the advantage of maintaining the adjuvant effect of the virosome associated saponins whilst showing a reduced reactogenicity as compared to non virosomal influenza formulations containing a saponin adjuvant. This patent application discloses use of sterol to maintain adjuvant effect of saponin and suggest using it in combination with saponin which has adjuvant effect.

“Nutritive Immune-enhancing Delivery System (NIDS) composed of combination of select vitamins, and a plant based polyphenol in various delivery systems including organic and inorganic pharmaceutically acceptable carriers are known to enhance both local and systemic immune responses in a mouse model following mucosal and systemic vaccinations with NIDS.” (J Vaccines Vaccin 2012, 3:4-74) Again, it suggests using polyphenol in combination with vitamins to enhance immune response. Here, polyphenol is disclosed in a very general term. A number of polyphenols are existing which are generally used as dietary supplements to enhance immunity. None of them has been evaluated as an adjuvant for the vaccine preparation against target antigen.

Prior art referred and discussed here suggest using polyphenol or sterol for maintaining of the adjuvant property of the known adjuvant used in the vaccine composition. It does not provide use of polyphenol or sterol as a sole adjuvant for enhancement of the immune response. Here, in the present invention, inventors have found out selected phytosterol, preferably beta-sitosterol as an adjuvant for enhancement of immune response against target antigen. Furthermore, adjuvant disclosed in the present invention is not limited to few antigens in providing higher immune response. It can provide higher immune response against variety of antigens such as peptide based antigen, recombinant antigen, virus-like particles based antigen, antigen in virosome form, etc.

“Numerous challenges remain related to adjuvant development. In effect, it is unlikely that any single immunostimulant or delivery system will be sufficient to induce the broad and long-lasting immunity that is required for all new vaccines. Effective adjuvant systems are likely to require synergy between one or more immunostimulants, and a carrier or delivery system. In addition, it is often impossible to compare adjuvants analyzed in different laboratories, or even within the same laboratory, because adjuvant formulation and characterization methods are not standardized. Furthermore, each antigen has a different intrinsic immunogenicity and interacts differently with immunostimulants and carriers, and no reliable algorithms exist to permit selection of optimal adjuvants based on physico-chemical or immunological properties of an antigen.” (Trends in Immunology, Vol. 30, No. 1, p 23-32) In such a situation, present invention provides single adjuvant which can provide surprisingly higher immune response against variety of antigens without any adverse effect. Furthermore, such adjuvant has synergy with other known adjuvant such as delivery system like IRIV and immunopotentiator such as alum. Thus, the present invention provides novel adjuvant system where there is synergy between novel adjuvant according to the present invention and another adjuvant. It is well known that the challenge for adjuvant system is to define the best combination for an effective and safe formulation in which individual components can synergize with one another to elicit a more robust immune response. Therefore, not all the known adjuvants can work with another known adjuvant effectively while, the present invention provides adjuvant system comprising novel adjuvant beta-sitosterol with another known adjuvant which can provide surprisingly higher immune response against target antigen.

### Virosomes:

Influenza virosomes are a clinically proven vaccine carrier/adjuvant system with an excellent safety and tolerability profile in humans. Influenza virosome as vaccine have been distributed in Europe Asia and America with over seventy millions doses distributed worldwide. The capability of the virosomal delivery system to mediate antigen processing through both the exogenous and the endogenous pathway makes this carrier a good candidate to test. The adjuvant properties of IRIVs are well known in the art, for example from WO 92/19267, wherein an adjuvant effect of the IRIVs for an antigen coupled thereto is disclosed.

However, although the use of virosomes as adjuvant has a number of advantages, for example low toxicity and high immunogenicity, one of the problems in current vaccinology is the lack of required immunogenicity of low immunogenic antigens. Therefore, combination of adjuvant which can enhance immune response together with the delivery system such as liposome or virosome is preferable. For subunit vaccines, it is highly desirable that the combination of delivery systems, immunopotentiators such as adjuvants and isolated antigens to elicit optimal immune responses. In many cases, the addition of additional adjuvants to the virosomal formulation destroy the immunological properties of the virosomal formulations due to high polarity of such adjuvants e.g. alum adjuvants deform the virosomes and squalene based adjuvants like MF-59 solubilizes the virosomal membrane. It depicts that there is difficulty in development of an adjuvant system comprising a delivery system and/or immunopotentiators.

Therefore, there is a need to develop an efficient immunopotentiating adjuvant system which can be used in the preparation of immunogenic composition and provide superior humoral and cellular immune response against the antigens of interest.

Here, in the present application, inventors have developed a novel combination of the phytosterols, preferably beta-sitosterol and immunostimulating reconstituted influenza virosomes (IRIV), without destroying the immunostimulating effect of each system. Surprisingly, such an adjuvant system showed a higher stimulating effect than virosome alone.

### OBJECT OF THE INVENTION

In a first aspect, the current invention provides certain polyphenol(s) as an adjuvant for the vaccine preparation against targeted antigen.

In a preferred aspect, the polyphenol(s) are preferably phytosterols such as flavonoids, more preferably Beta-sitosterol.

In a second aspect, the current invention provides a novel adjuvant system comprising certain polyphenols and a suitable delivery system. Preferably, polyphenols is beta-sitosterol and the delivery system is IRIV according to the present invention.

In a third aspect, the current invention provides an immunogenic composition comprising an antigen of interest along with the polyphenol(s) of the present invention.

In a further aspect, the current invention provides an immunogenic composition comprising antigen of interest and a combination of the polyphenol(s) of the present invention along with suitable delivery system such as IRIV as described herein.

In fourth aspect, the current invention provides an immunogenic composition comprising antigen of interest and a combination of polyphenol according to the present invention along with other known adjuvant(s) as a second adjuvant.

In one of the aspects, an antigen of interest includes infectious agent selected from a bacterium, a virus, a parasite and a fungus. In a preferred aspect, the antigen of interest is isolated fragment of virus or whole virus or isolated fragment of parasite or isolated fragment of fungus. Isolated fragment according to the present invention can be structural protein of antigen of interest.

In fifth aspects, the present invention provides a method of extraction of polyphenol(s), preferably flavonoids from plant source(s) such as vegetables or fruits.

In a sixth aspect, the present invention provides a method of preparing immunogenic composition comprising antigen of interest and polyphenol, preferably beta-sitosterol.

In a furthermore aspect, the present invention provides a method of preparing an adjuvant system comprising the polyphenols and a suitable delivery system. Preferably, the delivery system is IRIV according to the present invention.

In a seventh aspect, the present invention provides use of polyphenol(s) as an adjuvant for the development of vaccine against infectious agent or carcinogenic or pathogenic agents or target protein.

In an eighth aspect, the present invention provides use of an adjuvant system comprising virosome (IRIVs) and the phytosterol(s) for the development of vaccine against infectious agent or carcinogenic or pathogenic agents or target protein.

In a furthermore aspect, the present invention provides use of an adjuvant system comprising known adjuvant(s) as a second adjuvant and the phytosterol(s) for the development of vaccine against infectious agent or carcinogenic or pathogenic agents or target protein.

In a preferred aspect, the present invention provides combination of the adjuvant systemwith antigen to induce protection level of immune response.

In a ninth aspect, the present invention provides a pharmaceutical composition for inducing an immune response against an immunogenic molecule(an antigen of interest) comprising the immunogenic composition comprising the antigen of interest along with the adjuvants or adjuvant system of the present invention along with suitable pharmaceutically acceptable carrier(s) or excipient(s).

In a tenth aspect, the present invention provides a vaccine comprising the immunogenic composition of the present invention for various antigens. These vaccines can be administered in conventional routes and dosages.

In one of the aspects, the effective dose or effective amount of the antigen according to the present invention is 1 μg-1000 μg of antigen per human dose, preferably 1-500 μg of antigen per human dose, more preferably 5 μg-250 μg of antigen per human dose.

In a preferred aspects, the effective dose or effective amount of the recombinant antigen or VLP based antigen according to the present invention is 1 μg-500 μg of antigen per human dose, preferably 5 μg-80 μg of antigen per human dose, more preferably 5 μg-25 μg of antigen per human dose.

In another preferred aspects, the effective dose or effective amount of the antigenic peptide according to the present invention is 1 μg-500 μg of antigen per human dose, 50 μg-500 μg of antigen per human dose, more preferably 50 μg-250 μg of antigen per human dose.

In further aspect, the effective amount of beta-sitosterol according to the present invention is 1 μg-200 μg of beta-sitosterol per human dose, preferably 5 μg-100 μg of beta-sitosterol per human dose, more preferably 20 μg-50 μg of beta-sitosterol per human dose.

In another aspect, the effective amount of second adjuvant(s) according to the present invention is 1 μg-1000 μg of adjuvant per human dose, preferably 1 μg-900 μg of adjuvant per human dose, more preferably 2 μg-500 μg of adjuvant per human dose.

In another aspect, the effective amount of solution of second adjuvant(s) or delivery system according to the present invention is 0.01 ml to 5 ml of adjuvant solution or delivery system per human dose, preferably 0.02 ml to 2 ml of adjuvant solution or delivery system per human dose, more preferably 0.05 ml to 1 ml of adjuvant solution or delivery system per human dose.

In one of the embodiments, the present invention provides a method of stimulating immune response of a patient in need thereof comprising administering a suitable dosage of immunogenic composition as disclosed in the current invention.

### DETAILED DESCRIPTION OF THE FIGURES

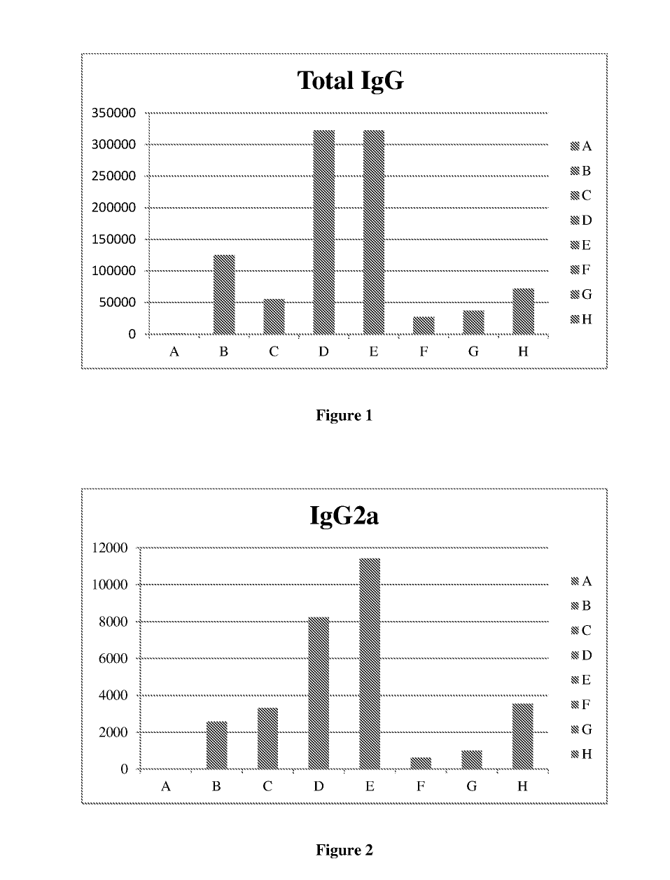

FIG. 1 depicts serum titers of total IgG antibody against Influenza A/Singapore/6/86(H1N1)

FIG. 2 depicts serum titers of IgG2a antibody against Influenza A/Singapore/6/86(H1N1)

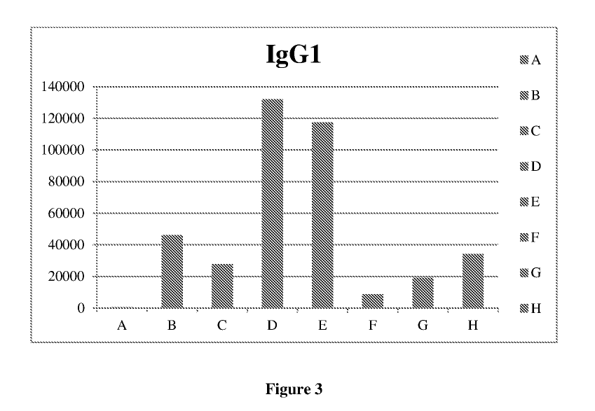

FIG. 3 depicts serum titers of IgG1 antibody against Influenza A/Singapore/6/86(H1N1)

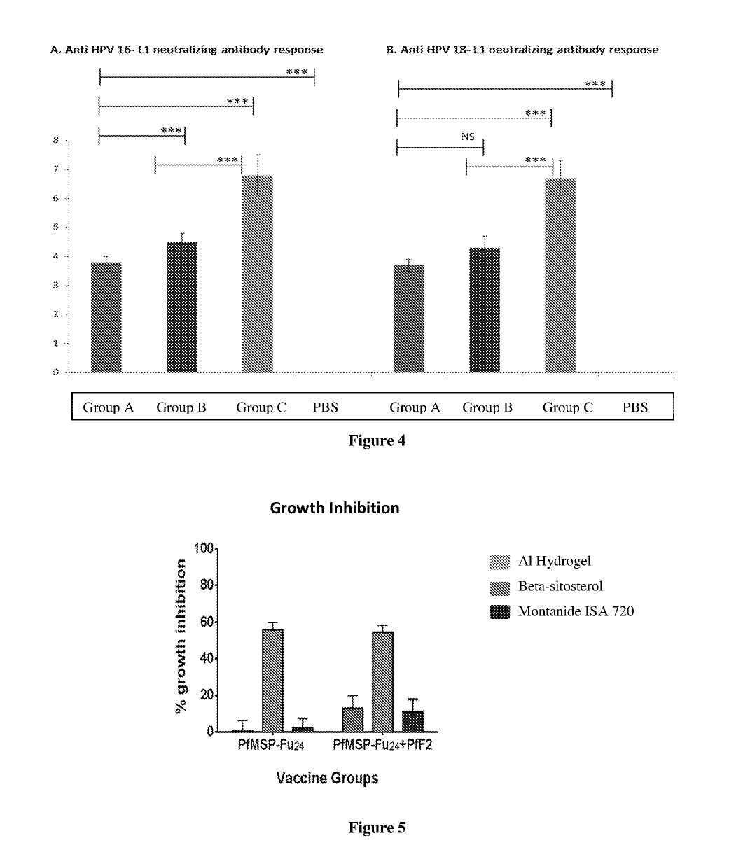

FIG. 4 depicts anti-HPV16L1 and anti-HPV18L1 neutralizing antibody titers elicited in mice immunized with Gardasil (Group A), HPV vaccine formulated with aluminum hydroxide (Group B), HPV vaccine formulated with beta-sitosterol (Group C) and PBS (Phosphate Buffer saline)- as the negative control. Statistical significance of the difference in titer between groups was determined by one-way non-parametric analysis of variance. P<0.05 was considered statistically significant. Significance was calculated with respect to the Gardasil group (* denotes level of significance while NS=non-significant). Antibody titer is expressed as log 10 IC50±S.D.

FIG. 5 depicts Growth-inhibitory activities of sera from mice immunized with PfMSPFu24+PfF2 or PfMSPFu24 formulated with human compatible adjuvants Al hydrogel, beta-sitosterol and Montanide ISA720.

### DETAILED DESCRIPTION OF THE INVENTION

The present invention is directed to use of certain polyphenol(s) as an adjuvant for vaccine preparation against targeted antigen.

Polyohenol(s) according to the present invention can be taken from natural source such as plants (citrus fruits or vegetables). It can be synthesized chemically also according to the present invention. The term ‘polyphenol’ or ‘phytosterol’ or ‘flavonoids’ can be used in place of each other according to the present invention. Polyphenols are phytochemicals, meaning compounds found abundantly in natural plant food sources that have antioxidant properties. There are over 8,000 identified polyphenols found in foods such as tea, wine, chocolates, fruits, vegetables, and extra virgin olive oil.

In one of the embodiments, the polyphenol(s) are preferably phytosterols such as sterols and other flavonoids. Phytosterol(s) can be selected from sitosterol, stigmasterol, campesterol, cholesterol and the like. Flavonoids can be selected from Flavones, Isoflavones, Flavonols, Catechins, Flavanones, Anthocyanins, Proanthocyanidins and the like. It is well known that phytosterols and other flavonoids are known to have immunomodulatory properties. Their applicability as nutritive supplement is also known in the art. But, all the sterols having immunomodulatory properties do not work as an adjuvant in vaccine preparation.

The current invention provides certain polyphenols preferably selected phytosterol as an adjuvant for the preparation of vaccine against target antigen. In a preferred embodiment, the polyphenols as per the present invention are selected from beta-sitosterol, campesterol, narigenin, neo hesperidin. In a more preferred embodiment, the polyphenol used as an adjuvant according to the present invention is beta-sitosterol.

In a specific embodiment, the present invention provides vaccine composition containing target antigen and polyphenols, preferably phytosterols. Such vaccine composition comprising selected phytosterol, preferably beta-sitosterol as an adjuvant provides surprisingly higher immune response as compared to other known phytosterols or IRIV (approved adjuvant for human vaccine) or alum (approved adjuvant for human vaccine) or any conventional adjuvant.

The polyphenol(s) or phytosterol of the present invention is suitable in vaccine preparation as effective and above all, provides safe vaccine. As we know, one of the major challenges in the field of adjuvant research is to select the adjuvant having higher potency while minimum toxicity.

In a preferred embodiment, the current invention provides vaccine composition comprising phytosterol with an antigen of interest which can provide higher immune response against target antigen with reduced toxicity when compared to the antigen itself.

In a more preferred embodiment, the current invention provides vaccine composition comprising beta-sitosterol and antigen of interest which can provide higher immune response against target antigen.

In another embodiment, the present invention provides novel adjuvant system comprising said phytosterol(s) and a suitable delivery system and/or a suitable adjuvant(s). The said adjuvant system developed according to the present invention provides surprisingly higher immune response against targeted antigen as compared to conventional adjuvants without affecting adjuvant property of the other component of the system.

Suitable delivery system according to the present invention can be virosome prepared from any antigen using standard method of virosome preparation. A virosome is a reconstituted viral envelope possessing membrane lipids and viral glycoproteins, but devoid of viral genetic information. Such virosomes are immunostimulating reconstituted influenza virosomes (IRIVs) or virosomes prepared from Respiratory Syncytial Virus (RSV virosome) or the like. The virosome can be produced from the RSV virus, by dissolving the RSV membrane, taking out the genetic material and reconstituting the lipid membrane including the natural RSV proteins.

Suitable adjuvants include Alum based adjuvants, mineral salt adjuvants, Complete Freund's adjuvant (CFA), Incomplete Freund's adjuvant (IFA), montanide, MF 59 and Adjuvant 65, bacterially derived adjuvants, lipophilic adjuvants, hydrophilic adjuvants and combination thereof. Mineral salt adjuvant is selected from salts of calcium, iron and zirconium or their suitable combination. Lipophilic adjuvant is selected from Telormedix, Mono Phosphoryl Lipid A (MPL), glucopyranosyl lipid adjuvant (GLA) or combination thereof.

In another embodiment, the present invention provides an immunogenic composition comprising an antigen of interest along with the polyphenol(s) of the present invention or adjuvant system comprising said phytosterol(s) and a suitable delivery system or a suitable adjuvant(s). Such an immunogenic composition induces protecting level of immune response against an antigen.

In a preferred embodiment, the present invention provides an immunogenic composition comprising an antigen of interest and beta-sitosterol or adjuvant system comprising beta-sitosterol and delivery system or suitable second adjuvant(s).

In a more preferred embodiment, the present invention provides an immunogenic composition comprising an antigen of interest and beta-sitosterol or adjuvant system comprising beta-sitosterol and IRIV or adjuvant system comprising beta-sitosterol and alum or GLA or MPL or others like.

Virosome either adsorb or incorporates an antigen of interest into itself to induce humoral or cellular response against an antigen of interest respectively.

### Virosome Preparation According to the Present Invention:

To obtain humoral immune response against an antigen of interest, first virosomes are formulated. In case of the lipophilic antigen, antigens are mixed with the formulated virosome; while in the case of hydrophilic antigen, antigens can be covalently linked to the surface of the virosome through cross-linkers, or entrapped in the virosomal structure.

We provide here IRIV preparation from influenza virus. IRIV is made up of three components: (a) a mixture of phospholipids; (b) essentially reconstituted functional virus envelopes; (c) an influenza hemagglutinin protein (HA) or a derivative thereof which is biologically active and capable of inducing the fusion of said IRIV with cellular membranes and of inducing the lysis of said IRIV after endocytosis by antigen presenting cells, preferably macrophages or B cells.

In a more preferred embodiment, the current invention provides an immunogenic composition comprising (a) a mixture of phospholipids; (b) essentially reconstituted functional virus envelopes; (c) an influenza hemagglutinin protein (HA) or a derivative thereof which is biologically active and capable of inducing the fusion of said IRIV with cellular membranes and of inducing the lysis of said IRIV after endocytosis by antigen presenting cells, preferably macrophages or B cells; and (d) Polyphenol preferably phytosterol as an adjuvant, preferably lipophilic adjuvant and (e) an antigen of interest.

The “mixture of phospholipids” described herein contains natural or synthetic phospholipids or a mixture thereof. At least it contains two different compounds selected from the group of glycero-phospholipids, such as phosphatidylcholine or phosphatidylethanolamine, and cholesterol.

The term “essentially reconstituted functional virus envelopes” refers to reconstituted influenza virus envelopes which are essentially devoid of the components which naturally occur inside of (below) the influenza virus envelope's membrane part. In a preferred embodiment the essentially reconstituted functional virus envelopes exhibit the form of a unilamellar bilayer. An example of such a lacking component is the matrix protein of the natural influenza virus envelope.

The term “biologically active HA or derivative thereof” as components of the IRIVs of the present invention refers to HAs or derivatives which substantially display the full biological activity of natural HA and are thus capable of mediating the adsorption of the IRIVs of the present invention to their target cells via sialic acid-containing receptors. Furthermore, such HA components can be recognized by circulating anti-influenza antibodies. This biological activity is an essential feature of the IRIVs of the present invention.

The term “lipophilic adjuvant” refers to TLR7 (Toll-like receptors) conjugated phospholipid i.e. Telormedix (herein after referred as TMX), Mono Phosphoryl Lipid A (herein after referred as MPL), GLA or combination thereof.

In one embodiment, an antigen of interest includes infectious agent selected from a bacterium, a virus, a parasite and a fungus. In a preferred aspect, the antigen of interest is isolated fragment of virus or whole virus or isolated fragment of parasite or isolated fragment of fungus. Isolated fragment according to the present invention can be structural protein of antigen of interest.

In another embodiment, an antigen of interest can be “antigenic peptide” which is derived from target protein and has ability to induce immune response in terms of antibody against the same target protein. For example, antigenic peptide against PCSK9 protein is antigen of interest according to the present invention. Such antigenic PCSK9 peptide which has ability to induce auto-anti-PCSK9 antibodies in a patient can be antigen of interest according to the present invention. WO 2011/027257 discloses such antigenic PCSK9 peptide or a functionally active variant thereof. An “antigenic peptide” as used herein can be linked to immunogenic carrier.

The term “immunogenic carrier” herein includes those materials which have the property of independently eliciting an immunogenic response in a host animal and which can be covalently coupled to a peptide, polypeptide or protein either directly via formation of peptide or ester bonds between free carboxyl, amino or hydroxyl groups in the peptide, polypeptide or protein and corresponding groups on the immunogenic carrier material, or alternatively by bonding through a conventional bifunctional linking group, or as a fusion protein. Examples of such immunogenic carriers are mentioned in WO 2011/027257.

In one of the embodiments, antigen of interest is virus-like particle (VLPs). As used herein, the term “virus-like particle” refers to a structure resembling a virus particle but which has been demonstrated to be non-pathogenic. In general, virus-like particles lack at least part of the viral genome. Also, virus-like particles can often be produced in large quantities by heterologous expression and can be easily purified. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. In a more preferred embodiment, VLP in accordance with the present invention is VLP of HPV virus or VLP of HEV virus or others like.

In another embodiment, antigen of interest is a recombinant antigen. It can be prepared by using recombinant technology.

In one of the embodiments, an antigen of interest according to the present invention includes Leishmania, Human Immunodeficiency virus (HIV), Hepatitis C virus (HCV), Hepatitis E virus (HEV), Hepatitis A virus (HAV), Hepatitis B virus (HBV), tuberculosis, herpes simplex virus (HSV), malaria causing parasites, Human Papilloma virus (HPV), PCSK9 peptide, influenza virus, measles virus, mumps virus, Ebola virus, Respiratory Syntial virus (RSV), West Nile virus (WNV) and others like. Here, antigen of interest can be isolated protein from such mentioned viral antigen. Preferably, isolated protein mentioned here can be structural proteins of the targeted virus.

Antigen of interest can be prepared by conventional methods or techniques which include sequentially cloning the gene of interest, expression of the gene of interest, purification and characterization of the protein obtained from the gene of interest. The steps mentioned herein above involve tools and techniques known in the art. A person skilled in the art can select such known techniques as per the requirement to achieve desired expression and purity of the antigen of interest.

The gene of interest can be isolated from the genomic DNA of the parasite using techniques available in the art such as DNA isolation, PCR technology, etc. or can be chemically synthesized. Cloning of gene of interest includes insertion of gene of interest into vector by using restriction enzyme at different cloning site. Vectors used in recombinant technology are known in the art.

Cloning is followed by transformation or transfection for further production of protein from the inserted gene of interest by using host cell system. The vector having gene of interest transforms or transfects it into host cell in which protein will be produced from inserted gene of interest.

Subsequently, high cell density fermentations can be carried out at the required scale by using methods known in the art. Such methods include batch, fed-batch and perfusion method. Here, in the present invention, fed-batch method is the preferred method for the large scale production of antigen of interest. Purification of protein obtained from the gene of interest is carried out by using column chromatography techniques or filtration techniques or suitable combinations thereof. Column chromatography techniques includes ion exchange column chromatography, hydrophobic interaction column chromatography, affinity column chromatography, size exclusion column chromatography, mixed mode column chromatography and combination thereof. Filtration techniques mainly include ultrafiltration and diafiltration using various buffers such as phosphate buffer, tris buffer, citrate buffers and others like. A person skilled in the art can select appropriate purification technique available in the art to achieve desired level of purity. Here, according to the present invention, ion exchange column chromatography technique is used to purify protein of interest, preferably protein of the target antigen.

In furthermore embodiment, the present invention provides a method of preparing an adjuvant system comprising of the polyphenols and suitable delivery system or suitable adjuvant. Preferably, the polyphenol, the delivery system and the suitable adjuvant is beta-sitosterol, IRIV and alum or GLA, respectively according to the present invention. In one of the embodiments, the present invention provides a method of extraction of polyphenol(s), preferably flavonoids from plant source(s) such as vegetables or fruits. Extraction method includes mainly following steps: (a) isolation of different organs of plant system; (b) extraction of plant oil from the isolated organ; (c) extraction of desired component preferably flavonoids or polyohenols from the extracted plant oil; (d) separation of the desired flavonoid or polyphenol.

Preferred plant system according to the present invention can be citrus plant more preferably citrus fruits. Seed, juice, pericarp, mesocarp or flavedo can be isolated for the extraction according to the present invention. Plant organs can be fresh material or stored material under different storage conditions. Preferred polyphenol according to the present invention can be beta-sitosterol. Various extraction methods such as solvent extraction, supercritical fluid extraction (SFE), microwave extraction or any other method which is known in the art can be used for the extraction of plant oil. These extraction methods are well known in the art. Here, in the present invention SFE is optimized in terms of parameters to improve the yield of the extract.

In one of the embodiments, the current invention provides a method of preparation of immunogenic composition comprising: (a) Preparation of antigen; (b) Preparation of beta-sitosterol or an adjuvant system

In a more preferred embodiment, the current invention provides a method of preparation of immunogenic composition comprising:
(a) Preparation of antigen;
(b) formulating a modified virosome with antigen or preparing mixture comprising second adjuvant and antigen;
(c) Addition of beta-sitosterol into modified virosome having antigen or into mixture comprising second adjuvant and antigen.
   In a preferred embodiment, the current invention provides a method of preparation of adjuvant system comprising:
(a) Formulation of modified virosome with antigen , preferably antigen is selected from lipophilic antien and hydrophilic antigen or preparing mixture comprising second adjuvant and antigen;
(b) Addition of polyphenol such as phytosterol into modified virosome having antigen or into mixture comprising second adjuvant and antigen.

Here, according to the present invention, polyphenol(s) are preferably phytosterols such as sterols and other flavonoids. Phytosterol(s) can be selected from sitosterol, stigmasterol, campesterol, cholesterol and others like according to present invention. Flavonoids can be selected from Flavones, Isoflavones, Flavonols, Catechins, Flavanones, Anthocyanins, Proanthocyanidins and others like according to present invention. In a more preferred embodiment, the phytosterol according to the present invention is beta-sitosterol. In another embodiment, the present invention provides use of the polyphenol(s) of the present invention as an adjuvant for the development of vaccine against infectious agent or carcinogenic or pathogenic agents.

In a preferred embodiment, the present invention provides use of beta-sitosterol as an adjuvant for the development of vaccine against infectious agent or carcinogenic or pathogenic agents.

In further embodiment, the present invention provides use of an adjuvant system comprising virosome and polyphenol, preferably phytosterol(s) to be used as an adjuvant for the development of vaccine against infectious agent or carcinogenic or pathogenic agents.

According to the present invention, beta-sitosterol is a preferred phytosterol to be used as an adjuvant.

In a preferred embodiment, the present invention provides combination of an adjuvant system with antigen to induce protection level of immune response.

In furthermore embodiment, the present invention provides use of an adjuvant system comprising conventional adjuvant and polyphenol, preferably phytosterol(s), more preferably beta-sitosterol as a polyphenol to be used for the development of vaccine against infectious agent or carcinogenic or pathogenic agents.

In one of the embodiments, the present invention provides a pharmaceutical composition for inducing an immune response against an immunogenic molecule (an antigen of interest) comprising immunogenic composition according to the present invention with pharmaceutically acceptable carrier or excipient. Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, microparticles, or in a liposomal preparation. Formulations for parenteral administration may be formulated to be immediate and/or modified release. Modified release formulations include delayed-, sustained-, pulsed-, controlled-, targeted and programmed release.

In a preferred embodiment, the present invention provides vaccine containing immunogenic composition of the present invention for various antigens. The vaccine comprises effective amount of an antigen of interest and immunogenic composition as disclosed in the current invention which can elicit an immune response against target antigen. These vaccines can be administered in conventional routes and dosages such as “pharmaceutically effective dose” or “therapeutically effective dose”.

An “effective amount” of an antigen of the invention, or composition thereof, is an amount that is delivered to a mammalian subject, either in a single dose or as part of a series, which is effective for inducing an immune response against target antigen in said subject. This amount varies depending upon the health and physical condition of the individual to be treated, the taxonomic group of individual to be treated, the capacity of the individual's immune system to synthesize antibodies, the formulation of the vaccine, and other relevant factors. It is expected that the amount will fall in a relatively broad range that can be determined through routine trials.

A “pharmaceutically effective dose” or “therapeutically effective dose” is that dose required to treat or prevent, or alleviate one or more antigen related disorder or symptom in a subject. The pharmaceutically effective dose depends on inter alia the specific compound to administer, the severity of the symptoms, the susceptibility of the subject to side effects, the type of disease, the composition used, the route of administration, the type of mammal being treated, the physical characteristics of the specific mammal under consideration such as health and physical condition, concurrent medication, the capacity of the individual's immune system to synthesize antibodies, the degree of protection desired, and other factors that those skilled in the medical arts will recognize. For prophylaxis purposes, the amount of peptide in each dose is selected as an amount which induces an immunoprotective response without significant adverse side effects in typical vaccines. Following an initial vaccination, subjects may receive one or several booster immunisations adequately spaced.

In one of the embodiments, the effective dose or effective amount of the antigen according to the present invention is 1 μg-1000 μg of antigen per human dose, preferably 1 μg-500 μg of antigen per human dose, more preferably 5 μg-250 μg of antigen per human dose. In a preferred embodiment, the effective dose or effective amount of the recombinant antigen or VLP based antigen according to the present invention is 1 μg-500 μg of antigen per human dose, preferably 5 μg-80 μg of antigen per human dose, more preferably 5 μg-25 μg of antigen per human dose.

In another preferred embodiment, the effective dose or effective amount of the antigenic peptide according to the present invention is 1 μg-500 μg of antigen per human dose, 50 μg-500 μg of antigen per human dose, more preferably 50 μg-250 μg of antigen per human dose.

In one of the embodiments, the effective amount of beta-sitosterol according to the present invention is 1 μg-200 μg of beta-sitosterol per human dose, preferably 5 μg-100 μg of beta-sitosterol per human dose, more preferably 20μg-50 μg of beta-sitosterol per human dose.

In another embodiment, the effective amount of second adjuvant(s) according to the present invention is 1 μg-1000 μg of adjuvant per human dose, preferably 1 μg-900 μg of adjuvant per human dose, more preferably 2 μg-500 μg of adjuvant per human dose. In one of the embodiments, the preferred adjuvant is alum or GLA or MPL or others like.

In another embodiment, the effective amount of solution of second adjuvant(s) or delivery system according to the present invention is 0.01 ml to 5 ml of adjuvant solution or delivery system per human dose, preferably 0.02 ml to 2 ml of adjuvant solution or delivery system per human dose, more preferably 0.05 ml to 1 ml of adjuvant solution or delivery system per human dose. In one of the embodiments, the preferred solution of adjuvant is montanide or virosome or MF59 or others like.

In one of the embodiments, the present invention provides a method of stimulating immune response of a patient in need thereof comprising administering a suitable dosage of immunogenic composition as disclosed in the current invention.

### Analytical Techniques Used in the Current Invention

ELISA: This is Enzyme linked sorbent assay where the seroconversion in the animals is measured by the interaction these antibodies have with the corresponding antigens. The results obtained are measured by the intensity of the color the reaction mixture develops after reacting with the substrate used in the reaction. The results are measured in ELISA units. HPV pseudovirus-based neutralization assay: It is performed as described in Vaccine 34 (2016) 4724-4731under section 2.5.2.
Growth inhibition test for malaria antigen: It is performed as described in PLoS ONE, October 2008, Volume 3, Issue 10, e3557, p1-10 under title CWRU (Growth inhibition Assays).

Extraction methods employed for the extraction of polyphenol such as beta-sitosterol from citrus: With reference to the extraction of components from citrus, different parts of fruit anatomy such as seed, juice, pericarp, mesocarp or flavedo can be used as a starting material for the extraction. Along with it, extraction from fresh material or stored material under different storage conditions can also be evaluated. Different storage conditions which can be employed for it are storage at room temperature, vacuum distillation, hydrolized water and vacuum distillation, hydro-cooling and vacuum distillation, etc.

In the present invention, one of the selected solutions was to extract the component oils obtained from flavedo. With reference to the storage condition of different parts of the fruit anatomy, the best method is combination of hydro-cooling and vacuum distillation. Hydro-cooling can be defined as the process or technique of arresting the ripening of fruits and vegetables after harvesting by immersion in ice water. Vacuum distillation can be defined as distillation of a liquid under reduced pressure, enabling it to boil at a lower temperature than normal.

Different extraction methods as mentioned below can be applied to oils obtained from flavedo or any other part of the fruit anatomy in order to extract flavonoids:

Solvent extraction method where solvent is ethanol, methanol or dimethyl formamide or other equivalent solvent can be employed for the extraction. Microwave extraction method provides an improvement in the yield of the extracted components as compared to solvent extraction method. In the present invention, supercritical fluid extraction (SFE) with CO₂ has been employed for the extraction of components identified in citrus oil including flavonoids, phenolic acids, and terpenes. The advantages of this method are biocompatibility, absence of even traces of solvents, reduced time for the extraction of the desired compound. The optimized parameters for the extraction according to the present invention are the following: vessel 100 ml; temperature of the vessel: 50° C.; temperature of micrometric valve 150° C.; Static phase: 40 min, Dynamic phase: 20 min; flow rate: 6L/min; pressure: 500 bars; Diatomaceous earth mixed with sample in 1:1-1:2 ratios. Time for the extraction: 300 minutes. Yield obtained by the extraction method having said optimized parameter is 4.1% w/w. It is superior to yield already published using supercritical fluid extraction (SFE) with CO₂ method (J. of Supercritical Fluids 55 (2010) 132-141).

### EXAMPLES

The following non-limiting examples describe the adjuvant system and its formulation with one of the antigen of interest which can be prepared as per the present invention. It will be appreciated that other immunogenic compositions with different antigens can be prepared and such immunogenic compositions are within the scope of a person skilled in the art and are to be included within the scope of the present invention.

### Example 1

### Preparation of IRIV

A pellet of purified influenza virus was solubilized using buffer and detergent system. The mixture was centrifuged and the supernatant containing the influenza spike proteins (HA) and viral phospholipids was added to the phospholipid mixture. The whole suspension was stirred for specific time. Subsequently, the suspension was submitted to batch chromatography in order to remove the detergent and to obtain the influenza virosome particles. This immunogenic composition was analyzed to determine the humoral immune response by conventional technique. The said analysis is denoted here as ‘Group H’ analysis.

### Example 2

### Preparation of Beta-Sitosterol as Adjuvant

Beta-sitosterol powder was prepared in a solution containing CMC 0.1-1%; Tween80® 0.1-1% and PBS. As alternative other detergents can be also used. The solution may contain also Squalene oil in a concentration of 1-10%. Solution is stirring at RT and then submitted to ultrasounds and kept at 4° C. till use. In an alternative method, Beta-sitosterol was extracted from different citrus fruits using conventional extraction methods as well as optimized supercritical fluid extraction (SFE) with CO₂ method. Conventional extraction methods or other optimized methods which can be employed according to the present invention for the extraction of the polyphenols such as beta-sitosterol are mentioned elsewhere in the specification.

### Example 3

### Preparation IRIV and Beta-Sitosterol Formulation

Just before the use the solution prepared as discussed in the Example 2 is submittedo several passages in microemulsion needle and then mixed with IRIV.

### Example 4

### Immunogenicity Study of Influenza Virosome Beta-Sitosterol

Groups of Balb/c mice 5 weeks old, had been immunized subcutaneously with the following formulations containing 1 μg of Influenza A virosome with or without flavonoids according to the below table 1. Three flavonoids formulated in the same way were analyzed: Hesperidin, Linoleic Acid Ethyl ester and Beta-sitosterol. 50 □l of formulation was administered in a two dose regimen at day 0 and 21.

TABLE 1
List of formulation prepared for immunogenicity
study of influenza virus vaccine

| Groups | No. of animals | Treatment |
|---|---|---|
| A | 5 | PBS pH 7.4 |
| B | 7 | Virosome + Hesperidin 1 μg |
| C | 7 | Virosome + Hesperidin 10 μg |
| D | 7 | Virosome + Beta sito sterol 1 μg |
| E | 7 | Virosome + Beta sito sterol 10 μg |
| F | 7 | Virosome + Linoleic acid Ethyl ester 1 μg |
| G | 7 | Virosome + Linoleic acid Ethyl ester 10 μg |
| H | 7 | Virosome without adjuvant |

Blood samples were collected after 35days and. for the analysis of humoral response by ELISA. Humoral response analysed by ELISA are shown in the graphical form as given in FIG. 1, FIG. 2 and FIG. 3. Data given in the figures clearly shows that beta-sitosterol provides higher immune response as compared to other analysed flavonoids or phytosterols and as compared to virosomes without adjuvant. This example also shows synergistic effect of the combination of beta-sitosterol and virosome preparation as an adjuvant system against influenza virus.

### Example 5

### Immunogenicity Study of HPV Vaccine Prepared with Beta-Sitosterol

Human papilloma vaccine comprising HPV16L1 and HPV18L1 antigens was prepared as described in Example 1 of WIPO publication of WO 2016/038625. Here, HPV16L1 and HPV18L1 antigens are prepared from the codon-optimized sequences as described in WO 2016/038625 (sequence ID 2 and sequence ID 3). HPV16L1 and HPV18L1 antigens can be prepared from any known nucleotide sequences encoding amino acid sequences of HPV16L1 and HPV18L1 antigens. Beta-sitosterol was isolated as mentioned in example 2 here using supercritical fluid extraction (SFE) with CO2 method from citrus fruit-orange. Four groups of mice for immunization study were designed in which mice of group A were immunized with Gardasil (approved HPV vaccine), mice of group B were immunized with HPV vaccine prepared as mentioned above and formulate with the aluminum hydroxide, mice of group C were immunized with HPV vaccine prepared as mentioned above and formulate with beta-sitosterol and PBS was administered to mice of group D as a negative control. Groups of Balb/c mice 5 weeks old, had been immunized subcutaneously with the following formulations as mentioned in the below table. Experimental design for immunogenicity studies in mice is described in below table 2. Blood samples were collected at day 28 for the analysis of hurnoral response by ELISA. Neutralizing antibody titers elicited after immunization was analysed by ‘HPV pseudovirus-based neutralization assay’ as described herein under ‘Analytical techniques’. FIG. 4 clearly shows that HPV vaccine formulated with beta-sitosterol provides surprisingly higher immune response against HPV antigens (HPV 16L1 and HPV18L1) as compared to the response obtained by the Gardasil and response obtained by the HPV vaccine formulated with the aluminum hydroxide. The results shown in FIG. 4 also depict that beta-sitosterol can work as a superior adjuvant in VLP based vaccine. Furthermore, administrated dose of HPV vaccine formulated with beta-sitosterol is half of the dose administered of approved HPV vaccine—Gardasil. It shows that beta-sitosterol can help us to reduce the dose amount which is substantial in terms of safety and toxicity parameters of the vaccine.

TABLE 2
Experimental design for immunogenicity study of HPV virus vaccine

| | | | | Amount | | |
|---|---|---|---|---|---|---|
| | | | | of | | |
| | | | Dose of | adjuvant | | |
| | | No. of | antigen | added | Equivalent | Day of |
| Group | | animals | (μg per | (μg per | human | immuni- |
| No. | Test groups | per group | mouse) | mouse) | dose | zation |
| A | Gardasil | 10 | 5 | NA | 1/8 | 0, 7, 21 |
| B | HPV | | 2.5 | 500 | 1/8 | 0, 7, 21 |
| | vaccine + | | | | | |
| | Aluminum | | | | | |
| | Hydroxide | | | | | |
| C | HPV | | 2.5 | 20 | 1/8 | 0, 7, 21 |
| | vaccine + | | | | | |
| | Beta- | | | | | |
| | sitosterol | | | | | |
| D | PBS | | NA | NA | 1/8 | 0, 7, 21 |

### Example 6

### Immunogenicity Study of Malaria Vaccine Prepared with Beta-Sitosterol

There were two different malaria antigen constructs prepared by using recombinant techniques for mentioned immunogenicity study. One of them was PfMSP-Fu₂₄ construct which was prepared as described in Indian application IN 1737/DEL/2008. Amino acid sequence of PfMSP-Fu24 malaria antigen is sequence ID: 1 as described in IN 1737/DEL/2008. Another construct PfF2 which was prepared as described in example of WIPO publication WO 2002/12292 or WO 2013/108272. Amino acid sequence of PfMSP-Fu24 malaria antigen is sequence ID: 17 as described in WO 2013/108272. Here, in the present study two separate malaria antigen preparations were made from these two malaria constructs. One had PfMSP-Fu₂₄ as malaria antigen and another had combination of PfMSP-Fu₂₄ and PfF2 (PfMSP-Fu₂₄+PfF2) as malaria antigen. The later preparation has been made as described in WIPO publication WO 2013/108272. Both the malaria antigen preparations were formulated with three different adjuvants to analyze the adjuvant effect of different formulations. Experimental design for immunogenicity studies in mice is described in below table 3. Here, beta-sitosterol was isolated as mentioned in example 2 here using supercritical fluid extraction (SFE) with CO2 method. Blood samples were collected at day 28 for the analysis of humoral response by ELISA. Growth inhibitory activities of mice sera against 3D7 P. falciparum strain obtained due to immunizations with the mentioned formulations was analysed by growth inhibition test as described in PLoS ONE, October 2008, Volume 3, Issue 10, e3557, p 1-10. Heat inactivated sera at 1:5 dilution was used in growth inhibition assay. Table 4 provided here below for the results obtained by the immunogenicity study as mentioned. FIG. 5 shows percentage (%) of growth inhibition in three different groups of mice immunized as mentioned in this example. FIG. 5 clearly shows that malaria vaccine formulated with beta-sitosterol provides surprisingly higher % of growth inhibition of 3D7 P. falciparum strain as compared to the % of growth inhibition by malaria vaccine formulated with aluminum hydroxide and montanide ISA 720.The results shown in FIG. 5 also depicts that beta-sitosterol can work as a superior adjuvant in recombinant vaccine.

TABLE 3
Experimental design for immunogenicity
study of malaria virus vaccine

| | | | | Amount of | |
|---|---|---|---|---|---|
| | | No. of | Dose of | adjuvant | |
| | | Animals | antigen | added | Day of |
| Antigen | | per | (μg per | (μg per | immu- |
| preparation | Adjuvant | group | mouse) | mouse) | nization |
| PfMSP-Fu₂₄ | Alum | 10 | 6 | 500 | 0, 7, 21 |
| | Beta-sitosterol | | | 5 | |
| | Montanide ISA | | | 70 μl | |
| | 720 | | | | |
| PfMSP- | Alum | | | 500 | |
| Fu₂₄ + PfF2 | Beta-sitosterol | | | 5 | |
| | Montanide ISA | | | 70 μl | |
| | 720 | | | | |

TABLE 4
Results obtained by growth inhibition assay

| | Adjuvant | | | | | |
|---|---|---|---|---|---|---|
| | | | | | Montanide | |
| | Alum | | Beta-sitosterol | | ISA720 | |
| Antigen | % of growth inhibition | | | | | |
| preparation | Mean | SD | Mean | SD | Mean | SD |
| PfMSP-Fu₂₄ | 0.38 | 5.88 | 55.8 | 4.076859 | 2.2 | 5.18 |
| PfMSP-Fu₂₄ + | 13 | 6.88 | 54.4 | 3.86 | 11 | 6.88 |
| PfF2 | | | | | | |

### Example 7

### Immunogenicity Study of PCSK9 Vaccine Prepared with Beta-Sitosterol

PCSK9 vaccine was prepared as described in WIPO publication WO 2011/027257. Here, ‘VR_9.5’ as decribed in WO 2011/027257 has been used as PCSK9 peptide for the current experiment. Other disclosed PCSK9 peptide can also be used as PCSK9 construct. The prepared PCSK9 construct was conjugated with diphtheria toxoid as an immunogenic carrier. The said preparation was used as an antigen preparation for further study. The conjugated PCSK9 antigen was formulated with alum and beta-sitosterol. Here, Beta-sitosterol was isolated as mentioned in example 2 here using supercritical fluid extraction (SFE) with CO2 method. Blood samples were collected at day 63 for the analysis of humoral response by ELISA. Humoral response analysed by ELISA is shown in below table. Experimental design for immunogenicity studies in mice is described in below table 5. Table 6 provided here below for the results obtained by ELISA.

TABLE 5
Experimental design for immunogenicity study of PCSK9
virus vaccine

| | | | Dose | Amount of | | |
|---|---|---|---|---|---|---|
| | | No. of | of | adjuvant | | |
| | | animals | antigen | added | | Day of |
| Group | | per | (μg per | (μg per | | immuni- |
| No. | Test groups | group | mouse) | mouse) | | zation |
| A | PBS | 10 | NA | NA | | 0, 15, 30 |
| B | Placebo 2 | | NA | Alum | 500 | |
| | (PBS + Alum + | | | Beta- | 20 | |
| | beta-sitosterol) | | | sitosterol | | |
| C | PCSK9 peptide + | | 300 | Alum | 500 | |
| | Alum + beta- | | | Beta- | 20 | |
| | sitosterol | | | sitosterol | | |

TABLE 6
Results obtained by ELISA

| Group No. | OD | ELISA Value | ELISA Titer |
|---|---|---|---|
| A | 0.048 | 2.764 | 55.28 |
| B | 0.0577 | 2.99 | 59.8 |
| C | 0.636 | 51.131 | 990.62 |

Results shown in table 6 clearly show that beta-sitosterol provides higher immune response against PCSK9 antigenic peptide as compared to GLA. Here, alum is used along with the beta-sitosterol. It depicts that beta-sitosterol can work with alum and other such adjuvants in combination and provides synergistic effect in terms of enhancement of the immune response.

1. An immunogenic composition comprising:
(a) an antigen;
(b) beta-sitosterol as an adjuvant.

2. The immunogenic composition as claimed in claim 1 further comprising of a delivery system or a second adjuvant.

3. The delivery system as claimed in claim 2 is virosome.

4. The virosome as claimed in claim 3 is an immunostimulating reconstituted influenza virosomes (IRIVs) or Respiratory Syncytial Virus virosome.

5. The adjuvant as claimed in claim 2, wherein the second adjuvant is selected from alum based adjuvants, mineral salt adjuvants, Complete Freund's adjuvant (CFA), Incomplete Freund's adjuvant (IFA), montanide, MF 59 and Adjuvant 65, bacterially derived adjuvants, lipophilic adjuvants, hydrophilic adjuvants or their suitable combinations.

6. The adjuvant as claimed in claim 5, wherein mineral salt adjuvants is selected from salts of calcium, iron and zirconium or their suitable combinations.

7. The adjuvant as claimed in claim 5, wherein lipophilic adjuvant is selected from Telormedix, Mono Phosphoryl Lipid A, glucopyranosyl lipid adjuvant and suitable combinations thereof.

8. The immunogenic composition as claimed in claim 2 comprising:
(a) beta-sitosterol;
(b) an immunostimulating reconstituted influenza virosomes or second adjuvant(s) (IRIVs).

9. A method of preparation of immunogenic composition as claimed in claim 1 comprising:
(a) preparation of antigen;
(b) formulating a modified virosome with antigen or preparing mixture comprising second adjuvant and antigen;
(c) addition of beta-sitosterol into modified virosome having antigen or into mixture comprising second adjuvant and antigen.

10. The method as claimed in claim 9, wherein the virosome is an immunostimulating reconstituted influenza virosomes (IRIVs).

11. The method as claimed in claim 9, wherein second adjuvant can be selected from alum based adjuvants, mineral salt adjuvants, Complete Freund's adjuvant (CFA), Incomplete Freund's adjuvant (IFA), montanide, MF 59 and Adjuvant 65, bacterially derived adjuvants, lipophilic adjuvants, hydrophilic adjuvants or their suitable combinations.

12. A pharmaceutical composition comprising immunogenic composition as claimed in claim 1, optionally with one or more pharmaceutically acceptable carrier or excipient capable of inducing an immune response against antigen.

13. A vaccine containing the immunogenic composition as claimed in claim 1 capable of inducing an immune response against antigen.

14. A method of stimulating immune response of a patient in need thereof comprising administering a suitable dosage of immunogenic composition according to claim 1.

15. The immunogenic composition as claimed in claim 1, wherein antigen is an infectious agent selected from a bacterium, a virus, a parasite and a fungus.

16. The immunogenic composition as claimed in claim 1, wherein antigen is a recombinant antigen, antigenic peptide or a virus-like particle.

17. The immunogenic composition as claimed in claim 1, wherein antigen is selected from Leishmania, Human Immunodeficiency virus (HIV), Hepatitis C virus (HCV), Hepatitis E virus (HEV), Hepatitis A virus (HAV), Hepatitis B virus (HBV), tuberculosis, Herpes Simplex virus (HSV), malaria causing parasites, Human Papilloma virus (HPV), PCSK9 peptide, influenza virus, measles virus, mumps virus, Ebola virus, Respiratory Syntial virus (RSV), West Nile virus (WNV) or a combination of any of the foregoing.

18. The antigen as claimed in claim 1 is present in the concentration range of 1 μg-1000 μg of antigen per human dose.

19. The immunogenic composition of claim 1, in which the beta-sitosterol is present in the amount of 1 μg-200 μg of beta-sitosterol per human dose.

20. The immunogenic composition of claim 2, in which the second adjuvant or the delivery system is present in the amount of 0.01 ml to 5 ml per human dose.

21. The immunogenic composition of claim 16, in which the recombinant antigen or virus-like particle is present in the concentration range of 1 μg-500 μg of antigen per human dose.
